(19) United States
(12) Patent Application Publication
(10) Pub. No.: US 2015/0093740 A1
(43) Pub. Date: Apr. 2, 2015

(54) INDEPENDENT PORTS IN AUTOMATED BIOREACTOR SYSTEM

(71) Applicant: WEYERHAEUSER NR COMPANY, FEDERAL WAY, WA (US)

(72) Inventor: Anthony P. Swanda, Snoqualmie, WA (US)

(73) Assignee: WEYERHAEUSER NR COMPANY, FEDERAL WAY, WA (US)

(21) Appl. No.: 14/501,584

(22) Filed: Sep. 30, 2014

Related U.S. Application Data
(60) Provisional application No. 61/884,837, filed on Sep. 30, 2013.

(51) Int. Cl. C12N 5/04; C12M 1/26; C12M 1/34

(52) U.S. Cl. CPC C12N 5/04; C12M 41/26; C12M 41/12; C12M 41/34; C12M 41/36; C12M 33/00
USPC 435/3; 435/287.1; 435/286.1; 435/420

(57) ABSTRACT

A totipotent plant tissue multiplication system can include a sterile enclosure and a bioreactor in the sterile enclosure. The bioreactor can include a number of ports. A sensor can be inserted into a first port. A matter insertion system can input matter into the bioreactor via tubing configured to be connected to a second port. The sensor can be removed from the first port during operation of the matter insertion system. The tubing can be disconnected from the second port during operating of the sensor and the culture transfer system.

### CROSS REFERENCE TO RELATED APPLICATIONS

This application is entitled to and claims the benefit of priority under 35 U.S.C. §119 from U.S. Provisional Patent Application Ser. No. 61/884,837 filed Sep. 30, 2013, and titled “INDEPENDENT PORTS IN AUTOMATED BIOREACTOR SYSTEM,” the contents of which are incorporated herein by reference.

The present application is related to U.S. application Ser. No. __/___,___ (attorney docket no. 27044-US-ULT), filed herewith and entitled “GAS CONTROL IN AUTOMATED BIOREACTOR SYSTEM,” and also related to U.S. application Ser. No. __/___,___ (attorney docket no. 27084-US-UTL), filed herewith and entitled “CULTURE HARVESTING IN AUTOMATED BIOREACTOR SYSTEM,” the contents of which are hereby incorporated by reference in their entirety.

### BACKGROUND

Many growers of plants desire to grow plants with particular characteristics. Replication of plants with desirable characteristics can be done using somatic cloning methods to produce numerous, genetically identical, clones of the particular plants. Such a replication may be of benefit with slow-maturing plant species, such as coniferous trees, including pines and firs. Rapid replication methods, such as somatic cloning can also provide an adequate supply of plants to identify individual plants that possess desirable characteristics.

Somatic cloning is the process of creating genetically identical plants from plant somatic tissue. Plant somatic tissue is plant tissue other than the male and female gametes. In one approach to somatic cloning, plant somatic tissue is cultured in an initiation medium which includes hormones, such as auxins and/or cytokinins, that initiate formation of embryogenic cells that are capable of developing into somatic embryos. The embryogenic cells are then further cultured in a maintenance medium that promotes multiplication of the embryogenic cells to form pre-cotyledonary embryos (i.e., embryos that do not possess cotyledons). The multiplied embryogenic cells are then cultured in a development medium that promotes development of cotyledonary somatic embryos. Cotyledonary somatic embryos can be placed, for example, within artificial seeds and sown in the soil where they germinate to yield conifer seedlings. The seedlings can be transplanted to a growth site for subsequent growth and eventual harvesting to yield lumber or wood-derived products. The cotyledonary somatic embryos can also be germinated in a germination medium, and thereafter transferred to soil for further growth.

### SUMMARY

Illustrative embodiments of the present application include, without limitation, methods, structures, and systems. In one embodiment, a totipotent plant tissue multiplication system can include a sterile enclosure and a bioreactor that includes a plurality of ports in the sterile enclosure. A sensor can be inserted into a first port. A matter insertion system can input matter into the bioreactor via a first tubing configured to be connected to a second port. The sensor can be removed from the first port during operation of the matter insertion system. The first tubing can be disconnected from the second port during operating of the sensor.

In one example, the sensor can include one or more of a dissolved gas sensor, a sugar sensor, a pH sensor, or a temperature sensor. The sensor can be in communication with a controller that is in communication with the matter insertion system. The matter insertion system can be configured to control input of the matter into the bioreactor based on signals from the sensor. The matter can include one or more of medium, acidic material, basic material, or gasses. If the matter includes cases, the first tubing can include a sterilization filter configured to sterilize gasses entering the bioreactor via the first tubing. The matter insertion system can include a pump configured to pump the matter into the bioreactor via the first tubing.

In another example, the system can further include a culture transfer system configured to either transfer culture into or harvest culture out of the bioreactor via a second tubing. The second tubing can be connected to a third port of the plurality of ports during operation of the at least one sensor and the matter insertion system. The culture transfer system can include a pump. The pump of the culture transfer system can be configured to pump culture from a culture source into the bioreactor via the second tubing. The pump of the culture transfer system can also be configured to pump culture out of the bioreactor via the second tubing and into a harvest container. The pump of the culture transfer system can also be configured to pump a sample of culture out of the bioreactor via the second tubing.

In another embodiment, a method of performing totipotent plant tissue multiplication can include inserting at least one sensor into a first port of a bioreactor, connecting a first tubing to a second port of the bioreactor, the first tubing being a part of a matter insertion system configured to input matter into the bioreactor via the first tubing, and performing totipotent plant tissue multiplication within the bioreactor while the bioreactor is located inside of a sterile enclosure. The method can also include performing one or more of the following during totipotent plant tissue multiplication within the bioreactor: removing the at least one sensor from the first port during operation of the matter insertion system, and disconnecting the first tubing from the second port during operation of the at least one sensor.

In one example, the method can also include attaching a second tubing to a third port of the bioreactor without affecting operation of the at least one sensor and the matter insertion system, the second tubing being a part of a culture transfer system configured to either transfer culture into or harvest culture out of the bioreactor via the second tubing. The method can also include transferring culture from the bioreactor to a harvest container via the second tubing while the harvest container and the bioreactor are located within the sterile enclosure. The method can also include closing the harvest container while is located within the sterile enclosure. The method can also include removing the closed harvest container from the sterile enclosure. The method can also include weighing the culture transferred from the bioreactor into the harvest container. The method can also include transferring a portion of the culture from the harvest container into the bioreactor via the second tubing after weighing the culture, and performing totipotent plant tissue multiplication within the bioreactor using the portion of the portion of the culture transferred from the harvest container into the bioreactor. The method can also include controlling the matter insertion system during totipotent plant tissue multiplication within the bioreactor based on signals from the at least one sensor.

### BRIEF DESCRIPTION OF THE DRAWINGS

Throughout the drawings, reference numbers may be re-used to indicate correspondence between referenced elements. The drawings are provided to illustrate example embodiments described herein and are not intended to limit the scope of the disclosure.

FIG. 1 depicts an embodiment of a bioreactor system.

FIGS. 2A, 2B, and 2C depict top views of embodiments of rocker pans with bioreactors placed on top.

FIGS. 3A, 3B, and 3C depict side views of a rocker with a pan and two bioreactors located on the pan.

FIG. 4 depicts a bioreactor system that includes control of multiple rockers.

FIG. 5 depicts an embodiment of a gas source and control system.

FIG. 6 depicts an embodiment of a method of providing gases to bioreactors in a cyclical format.

FIG. 7 depicts an embodiment of a system of gas sources.

FIG. 8 depicts an example of a pH control system inside of a bioreactor system.

FIG. 9 depicts a bioreactor system 900 with a control system for controlling an amount of multiplication medium that is fed into bioreactors.

FIGS. 10A and 10B depict examples of ports used with bioreactors.

### DETAILED DESCRIPTION OF ILLUSTRATIVE EMBODIMENTS

The multiplication (maintenance) stage of somatic cloning of plant tissue in the laboratory is typically carried out in liquid suspension cultures in shake flasks using a batch method, also known as splitting. In the practice of a batch culture method, embryogenic tissue is cultured in liquid multiplication medium for a period of time; the embryogenic tissue is separated from the multiplication medium (e.g., by allowing the embryogenic tissue to settle out of the medium); then aliquots of the embryogenic tissue are removed and introduced into separate volumes of fresh multiplication medium for further culture. This process is repeated as often as desired to yield a multiplicity of containers that each include separate batches of the embryogenic tissue culture. In addition to small volumes and multitudes of containers, it is difficult to control the growth conditions in shake flasks, and there is culture variability between flasks.

As used herein, “totipotent” refers to a capacity to grow and develop into a normal plant. Totipotent plant tissue has both the complete genetic information of a plant and the ready capacity to develop into a complete plant if cultured under favorable conditions. As is generally known in the art, totipotent plant tissue is obtainable from any of several areas of a plant, such as meristematic tissue and plant embryonic tissue.

Although the batch culture method is useful at laboratory scale, it is impractical to use the batch method for commercial-scale production of totipotent plant tissue. Moreover, the batch method is labor intensive and difficult to research the effects of multiple variables in the production of totipotent plant tissue. Bioreactors are more suitable for large-scale production and provide several advantages over the shake-flask, batch method, including automation, and the ability to more closely monitor and control the culture environment, such as sugar concentration, dissolved oxygen, carbon dioxide, and pH, resulting in more homogeneous cultures and higher yield of quality somatic embryos than the shake-flask method.

The successful operation of a bioreactor system for research purposes can include the automatic control of multiple bioreactors within the system. Each bioreactor can independently and quickly multiply totipotent plant tissue. Each bioreactor for multiplying totipotent plant tissue can be a fed-batch bioreactor, where a small volume of totipotent plant tissue and multiplication media is inoculated into the bioreactor and additional multiplication media is added over time until a sufficient volume of totipotent plant tissue (biomass) has been achieved or the maximum volume of the bioreactor is reached. Benefits of having such a research system can include process optimization of totipotent plant tissue multiplication, media optimization of totipotent plant tissue multiplication, liquid establishment of cultures, and genotype screening.

A number of variables in a bioreactor system can affect multiplication of totipotent plant tissue within individual bioreactors. For example, the rate of totipotent plant tissue growth can be affected by biomass concentration and the concentration of media components and extra cellular products in the bioreactor. Some media components, such as concentrations of sugar and plant hormones, can have an effect that is inversely proportional to biomass. Other variables, such as temperature, agitation rate, dissolved oxygen, pH, and carbon dioxide dosing rate, can affect multiplication of totipotent plant tissue within each bioreactor.

Production of totipotent plant tissue can also require a sterile environment. In some cases, production of somatic embryos can be done is a “clean room” that has been designed to have low levels of pollutants, such as dust, airborne microbes, and the like. Creating a clean room environment can be difficult as the air in the clean room must be filtered, contaminants must be removed, and the like. Entrance into a clean room typically requires an air-lock system that filters dust, particulate, and other contaminants from the air and allows people to put on suits that will maintain the clean room environment. People operating in clean rooms typically have to be fully covered in suits, limiting mobility and vision while working. In some cases, a sterile environment can be created in an open workspace, such as a work bench, with a hood that forces sterile air down onto the workspace to prevent contaminants from entering the work area. However, open workspaces can be contaminated by workers without proper training or care while working.

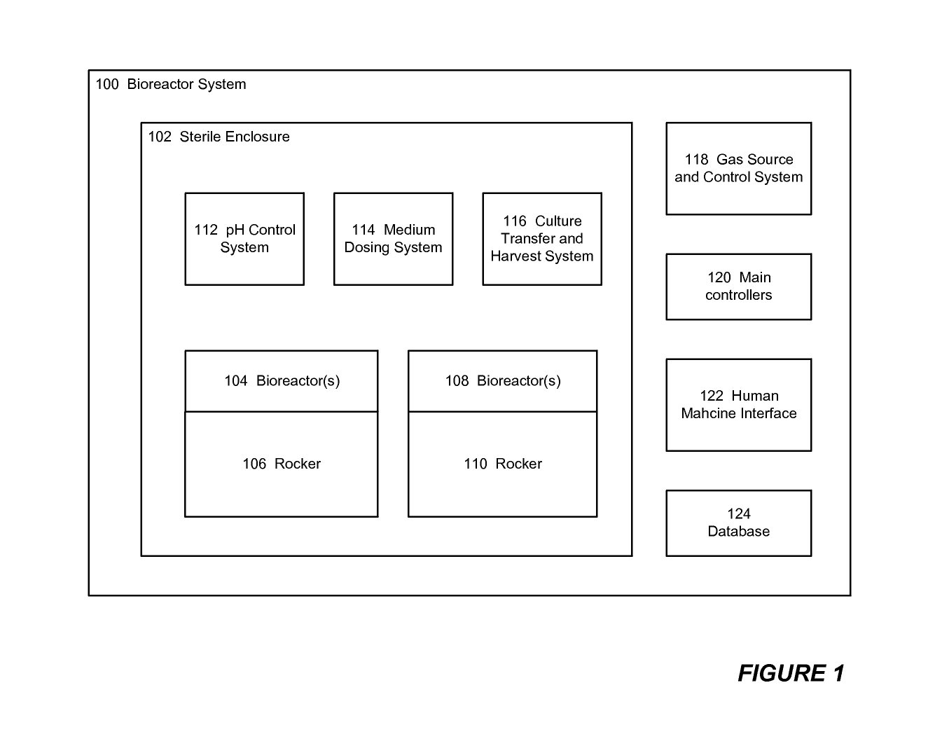

FIG. 1 depicts an embodiment of a bioreactor system 100. The bioreactor system 100 can include a sterile enclosure 102. The enclosure can include a hood to force sterile air down from the top of the sterile enclosure 102. The sterile enclosure 102 can include walls and a bottom that maintain a sterile atmosphere. The sterile enclosure 102 can also include one or more doors in the walls that can allow access to any of the components within the sterile enclosure 102. In operation, such doors may normally be closed while the bioreactor system automatically maintains certain conditions of totipotent plant tissue multiplication. The doors may be opened for limited times to adjust components or conditions within the sterile enclosure 102. However, limiting the number of times and the overall length of time that the doors are opened limits the risk of contamination within the sterile enclosure 102. The walls, bottom, and doors of sterile enclosure can be made of any rigid materials, such as transparent plastic or any other materials. The sterile enclosure 102 can include shades that can cover the walls to limit the amount of light that enters the sterile enclosure 102.

The bioreactor system 100 can also include one or more bioreactors 104 located on a first rocker 106 and one or more bioreactors 108 located on a second rocker 110. The first and second rockers 106 and 110 can be WAVE Base 20/50 rockers made by GE Healthcare Life Sciences. In one embodiment, each of the first and second rockers 106 and 110 can hold between one and four bioreactors. In that embodiment, the bioreactor system 100 can operate between one and eight bioreactors at any given time. While the system depicted in FIG. 1 includes first and second rockers 106 and 110, the first and second rockers 106 and 110 can be substituted with any other type of agitators. An agitator can be configured to agitate culture within one or more of the bioreactors 104 and 108. Various types of agitators include rockers, shake plates, culture mixers, culture stirrers, and the like. Throughout this disclosure, where a rocker or rockers may be described, other forms of agitators may be substituted as desired.

The bioreactors 104 and 108 can be made of a flexible plastic material in the form of a bag. The volume of the bioreactors 104 and 108 can be in a range from about 50 ml to about 10 L. The bioreactors 104 and 108 can include ports for connecting tubing and sensors to each bioreactor. The rockers 106 and 110 can include a pan upon which the bioreactors 104 and 108 can be placed. The rockers 106 and 110 can control movement of the pan to rock back and forth from one angle to another angle to cause wave-like movement of liquid in bioreactors 104 and 108. The rockers 106 and 110 can include a heating element to heat portions of the pan. The rockers 106 and 110 can also include a control system that controls the angle of rocking, the speed of rocking, and the temperature of the pan.

The bioreactor system 100 can include a pH control system 112. The pH control system 112 can regulate the level of pH within each bioreactor. The pH control system 112 can include a pH sensor in one or more of the bioreactors 104 and 108, a source of acidic material, a source of base, tubing connecting the source of acidic material and the source of basic material to each of the bioreactors 104 and 108, a peristaltic pump on each line of tubing between the source of acidic material and each of the bioreactors 104 and 108, a peristaltic pump on each line of tubing between the source of basic material and each of the bioreactors 104 and 108, and a controller configured to control the peristaltic pumps such that the pH within each of the bioreactors 104 and 108 is maintained based on a particular pH set point. A particular set point could be a particular value, a range of values, a particular value with an acceptable deviation, and the like. For example, in the case of bacterial or mammalian embryogenic tissue multiplication, a particular pH set point may be a range between about 6 and 8; for totipotent plant tissue multiplication, a particular pH set point may be a range between about 5.0 and 5.7; and for some Loblolly Pine cultures, a particular pH set point may be a pH level of about 5.5.

The bioreactor system 100 can include a medium dosing system 114. The medium may be formulated to promote the growth and multiplication of the embryonal suspensor masses. The medium may include hormones, such as auxins (e.g., 2,4-dichlorophenoxyacetic acid (2,4-D)) and cytokinins (e.g., 6-benzylaminopurine (BAP)). Auxins can be utilized, for example, at a concentration of from 1 mg/L to 200 mg/L. Cytokinins can be utilized, for example, at a concentration of from 0.1 mg/L to 50 mg/L. The medium may contain nutrients that sustain the totipotent plant tissue. It is generally desirable, though not essential, to include maltose as the sole, or principal, metabolizable sugar source in the medium. Examples of maltose concentrations may be within the range of from about 2.5% to about 6.0%. The osmolality of the medium may be in the range of 100-250 mM/kg. The medium dosing system 114 may include a sugar concentration sensor in one or more of the bioreactors 104 and 108, a source of medium, tubing connecting the source of medium to each of the bioreactors 104 and 108, a peristaltic pump on each line of tubing between the source of medium and each of the bioreactors 104 and 108, and a controller configured to control the peristaltic pumps such that the sugar concentration within each of the bioreactors 104 and 108 is maintained based on a particular sugar set point, such as about 3% sugar concentration. The medium dosing system 114 can also be an off-line system. In an off-line medium dosing system, a controller can be configured to pump a particular dose of medium into each of the bioreactors 104 and 108 at a particular dosing rate. Samples of the culture in each of the bioreactors 104 and 108 can be harvested on occasion and the sugar concentration of the samples can be determined. A user can adjust the dose amount and/or the dosing rate controlled by the controller based on the sugar concentration of the samples. The adjustments of the dose amount and/or the dosing rate can be received as inputs into the human machine interface 122 and sent to a controller of the medium dosing system 114. Alternatively, a user could determine a sugar concentration of the harvested samples and enter that determined sugar concentration into the human machine interface 122. A controller can calculate a particular dose or doses of medium into each of the bioreactors 104 and 108 at a particular dosing rate based on the sugar concentrate input by the user. The medium dosing system 114 can pump medium into each of the bioreactors 104 and 108 based on the particular dose or doses of medium and the particular dosing rate. Moreover, medium dosing can be added at non-linear amounts and rates, such as by exponentially increasing dose amounts, exponentially increasing dose rates, and the like.

The bioreactor system 100 can include a culture transfer and harvest system 116. Before multiplication begins, a culture of plant somatic tissue can be transferred into one of the bioreactors 104 and 108. The culture transfer and harvest system 116 can gravimetrically determine the volume of the culture transferred into one of the bioreactors 104 and 108. During or after the multiplication process, plant somatic tissue can be harvested out of one of the bioreactors 104 and 108. The culture transfer and harvest system 116 can gravimetrically determine the volume of the harvested plant somatic tissue. The culture transfer and harvest system 116 can include equipment for manually transferring culture into a bioreactor and harvested out of a bioreactor. The culture transfer and harvest system 116 can also include components for automatically transferring culture into a bioreactor and harvested out of a bioreactor.

To harvest culture from a bioreactor of the bioreactors 104 and 108, a port of the bioreactor can be clamped. A cap on the clamped port can be removed while the portion of the bioreactor with the port is located within the sterile enclosure 102. Sterile tubing can be attached to the port inside of the sterile enclosure 102. Keeping the portion of the bioreactor with the port inside of the sterile enclosure 102 while the cap is exposed can ensure that environment within the bioreactor remains sterile. After the tubing is attached to the port, the port can be unclamped and a pump connected to the tubing, such as a peristaltic pump, can pump culture out of the bioreactor and into a harvest container. Before or during the pumping of the culture out of the bioreactor, the bioreactor can be placed at an angle with the port at a lower end of the bioreactor such that the culture inside of the bioreactor tends to flow toward the port. If the bioreactor is located on a rocker in the sterile enclosure, the rocker could be placed at an angle with the port of the bioreactor at a lower position.

The harvest container can be weighed before and after the harvesting process to determine the weight of the harvested culture. The weighing of the harvest container can be done by a weighing device, such as a mass balance. The weighing device can be connected to a controller. The controller can receive an indication of the weight of the harvested culture (or indications of the weight of the harvest container before and after the harvesting process) from the weighing device. The controller can store information in a database about the harvested culture. The stored information can include a weight of the harvested culture. The controller can also estimate a mass of the harvested culture using the specific weight of water as an approximation for the specific weight of the harvested culture. The stored information can include the estimated mass of the harvested culture.

Once the pumping of culture out of the bioreactor is complete, the tubing can be removed from the port and the port can be closed. The port can be clamped before the tubing is removed and the port can be closed before the clamp is removed. If some totipotent plant tissue remains in the bioreactor after the harvesting process, multiplication of the totipotent plant tissue in the bioreactor can be resumed. The harvest culture container can also be closed while the harvest culture container is inside of the sterile enclosure, and then the closed harvest culture container can be removed from the sterile enclosure. After the closed harvest culture container is removed from the sterile enclosure, it can be moved to other non-sterile environments without disturbing the sterility of the environment within the closed harvest culture container. In one example, the closed harvest culture container can be moved to a laminar flow hood for further processing of the harvested culture, such as development of tissue or embryos.

Having a culture transfer and harvest system 116 that allows for harvesting of culture from bioreactors inside of a sterile enclosure can have a number of benefits. In one example, the bioreactor does not need to be removed from the sterile enclosure to harvest culture. This reduces the risk of contamination inside of the bioreactor during the harvesting process. In another example, the bioreactor does not need to be disconnected from other systems used in the multiplication process (e.g., a gas source and control system, a pH control system, a medium dosing system, etc.) in order for the harvesting to occur. This can save labor costs and time in the harvesting process.

A culture transfer system can transfer culture into a bioreactor. To transfer culture into a bioreactor, tubing can be connected from a source of culture to a port on a bioreactor. Attaching the tubing to a port on the bioreactor can include clamping the port, removing a cap, attaching the tubing to the uncapped portion of the port, and unclamping the port. A pump, such as a peristaltic pump, can be located along the tubing and configured to pump culture from the source of culture into the bioreactor. The source of culture can be weighed by a weighing device, such as a mass balance, both before and after culture is transferred from the source of culture to the bioreactor. The weighing device can be connected to a controller. The controller can receive an indication of the weight of the culture transferred into the bioreactor (or indications of the weight of the culture source before and after the transferring process) from the weighing device. The controller can store information in a database about the transferred culture. The stored information can include a weight of the transferred culture. The controller can also estimate a mass of the transferred culture using the specific weight of water as an approximation for the specific weight of the transferred culture. The stored information can include the estimated mass of the transferred culture.

In one embodiment, when culture is harvested from a bioreactor, as much of the culture as possible can be harvested from the bioreactor into a harvest culture container by the culture harvest system. The culture harvested from the bioreactor into the harvest culture container can be weighed to determine a final weight of the culture developed in the bioreactor. An agitator can be used to keep the harvested culture in the harvest culture container mixed. Keeping the harvested culture mixed can be helpful if the bioreactor is to be inoculated with a portion of the harvested culture. If the bioreactor is to be inoculated with a portion of the harvested culture, a culture transfer system can transfer some of the harvested culture in the harvest culture container back into the bioreactor. The weight of the harvest culture container can be weighed before and after the culture is transferred back into the bioreactor. The same pump and tubing can be used to harvest culture from the bioreactor and to transfer culture back into the bioreactor.

While the pH control system 112, the medium dosing system 114, and the culture transfer and harvest system 116 are depicted inside of the sterile enclosure 102 of the bioreactor system 100 in FIG. 1, some or all of the pH control system 112, the medium dosing system 114, and the culture transfer and harvest system 116 can be located outside of the sterile enclosure 102. For example, sources of acidic material, basic material, medium, and culture can all be located outside of the sterile enclosure 102. Those sources can be sealed containers that maintain their contents in a sterile condition. The sealed containers may include a venting filter, though venting filters may not be needed for disposable sealed containers. If any sealed container is outside of sterile enclosure 102, a sterile connection (such as sterile tubing) can connect the sealed container to the bioreactors 104 and 108 by passing through the boundaries of the sterile enclosure 102. In such cases, pumps (such as peristaltic pumps) for pumping material from the sealed containers to the bioreactors 104 and 108 can be located along the tubing either inside of the sterile enclosure 102 or outside of the sterile enclosure 102.

The bioreactor system 100 can include a gas source and control system 118. The gas source and control system 118 can be located outside of the sterile enclosure 102, as shown in FIG. 1. The gas source and control system can include sources of different types of gas, such as air, oxygen, nitrogen, and carbon dioxide. The sources of gas can provide sterilized gases, such as air, oxygen, nitrogen, and carbon dioxide. The sources of gas can be stand-alone sources, such as pressurized gas cylinders, or gas generators, such as generators that isolate certain types of gases from air. The gas source and control system 118 can control an amount of air provided to each of the bioreactors 104 and 108 based on dissolved gas sensors in each of the bioreactors 104 and 108. Dissolved gas sensors can include dissolved oxygen sensors, carbon dioxide sensors, and the like. The gas source and control system 118 can provide gases to each of the bioreactors 104 and 108 on a cyclical basis.

The bioreactor system 100 can include a main controller 120 that can be configured to control various aspects of the bioreactor system 100. In one embodiment, the main controller 120 can directly control portions of the other system in bioreactor system 100. For example, the main controller 120 can receive signals from sugar concentration sensors in the bioreactors 104 and 108 and send control signals to peristaltic pumps in the medium dosing system 114 that control the amount of medium that is pumped into each of the bioreactors 104 and 108. In another embodiment, the main controller 120 can send signals to individual controllers in other system in bioreactor system 100. For example, the main controller 120 may send an input to a controller of one of the rockers 106 and 110 indicating a desired angle and a desired rocking rate. The controller of the one of the rockers 106 and 110 can be configured to control the rocking motion of pan in accordance with the desired angle and a desired rocking rate. The main controller 120 can include one or more types of controllers, such as a programmable logic controller.

The bioreactor system 100 can include a human machine interface 122. The human machine interface 122 can include a computing system with software operating that allows a user to input various system controls. The software can be any software that can interface with main controller 120, such as Wonderware® InTouch® human machine interface software. The bioreactor system 100 can also include a database 124 that can store information about various bioreactor configurations and experiments. Data may be entered into database 124 manually using the human machine interface 122 or automatically by main controller 120. The data in database 124 may be useful to determine trends of experiments and optimal conditions for totipotent plant tissue multiplication. In one embodiment, the data in database 124 can be accessed, trends from data in the database 124 can be viewed, and optimal conditions for totipotent plant tissue multiplication can be viewed using the human machine interface 122.

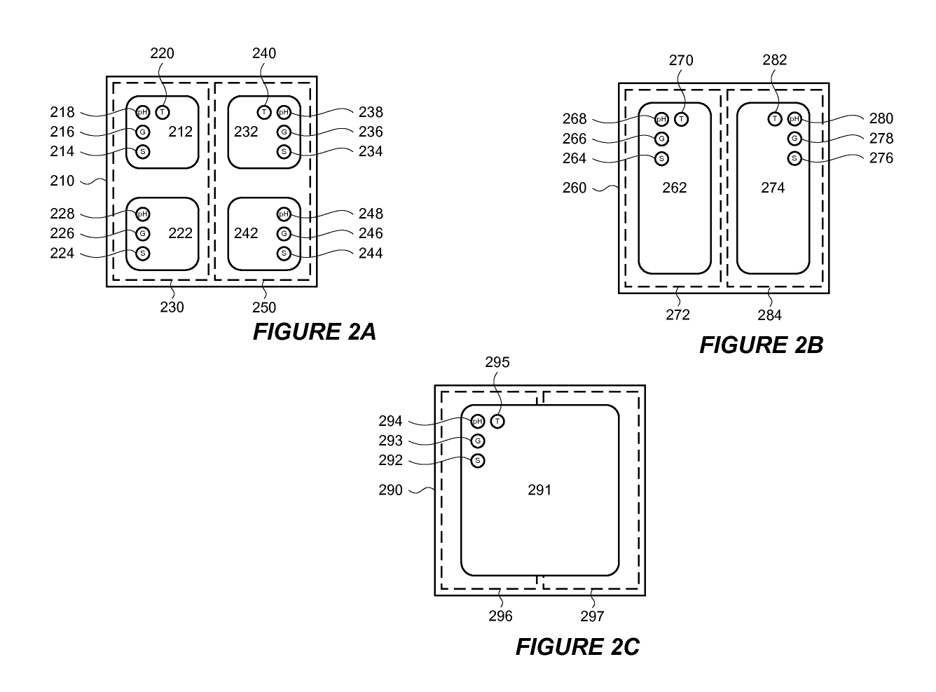

FIGS. 2A, 2B, and 2C depict top views of embodiments of rocker pans with bioreactors placed on top. FIG. 2A depicts a rocker pan 210 and bioreactors 212, 222, 232, and 242 placed on rocker pan 210. Bioreactor 212 includes a sugar concentration sensor 214, a dissolved gas sensor 216, a pH sensor 218, and a temperature sensor 220. The sensors 214, 216, 218, and 220 can be configured to sense conditions within the bioreactor 212. Bioreactor 222 includes a sugar concentration sensor 224, a dissolved gas sensor 226, and a pH sensor 228. The sensors 224, 226, and 228 can be configured to sense conditions within the bioreactor 222. Both bioreactors 212 and 222 are located over a single heating element 230 that is located in the pan 210. Since both bioreactors 212 and 222 are located over a single heating element 230, control of the heating element 230 can be based on a single temperature sensor 220. Thus, temperature sensors are not required to be in both bioreactors 212 and 222. Bioreactor 232 includes a sugar concentration sensor 234, a dissolved gas sensor 236, a pH sensor 238, and a temperature sensor 240. The sensors 234, 236, 238, and 240 can be configured to sense conditions within the bioreactor 232. Bioreactor 242 includes a sugar concentration sensor 244, a dissolved gas sensor 246, and a pH sensor 248. The sensors 244, 246, and 248 can be configured to sense conditions within the bioreactor 242. Both bioreactors 232 and 242 are located over a single heating element 250 that is located in the pan 210. Since both bioreactors 232 and 242 are located over a single heating element 250, control of the heating element 250 can be based on a single temperature sensor 240. Thus, temperature sensors are not required to be in both bioreactors 232 and 242.

FIG. 2B depicts a rocker pan 260 and bioreactors 262 and 274 placed on rocker pan 260. Bioreactor 262 includes a sugar concentration sensor 264, a dissolved gas sensor 266, a pH sensor 268, and a temperature sensor 270. The sensors 264, 266, 268, and 270 can be configured to sense conditions within the bioreactor 262. Bioreactor 262 is located over a single heating element 272. Control of the heating element 272 can be based on signals generated by temperature sensor 270. Bioreactor 274 includes a sugar concentration sensor 276, a dissolved gas sensor 278, a pH sensor 280, and a temperature sensor 282. The sensors 276, 278, 280, and 282 can be configured to sense conditions within the bioreactor 274. Bioreactor 274 is located over a single heating element 284. Control of the heating element 284 can be based on signals generated by temperature sensor 282.

FIG. 2C depicts a rocker pan 290 and a bioreactor 291 placed on rocker pan 290. Bioreactor 291 includes a sugar concentration sensor 292, a dissolved gas sensor 293, a pH sensor 294, and a temperature sensor 295. The sensors 292, 293, 294, and 295 can be configured to sense conditions within the bioreactor 291. Bioreactor 291, due to its size, is located over portions of two heating elements 296 and 297. Heating elements 296 and 297 can be controlled separately based on signals generated by temperature sensor 295.

A bioreactor system, such as the bioreactor system 100 depicted in FIG. 1, can include more than one rocker. In a bioreactor system with multiple rockers, the number of bioreactors on each rocker pan can be different. For example, in a two-rocker bioreactor system, one of the rockers can include four bioreactors, such as in the embodiment depicted in FIG. 2A, and the other of the rockers can include two bioreactors, such as in the embodiment depicted in FIG. 2B. Additionally, other configurations of bioreactors on a pan are possible. For example, a rocker pan can include two bioreactors over one heating element, such as in the case of bioreactors 212 and 222 located over heating element 230 in FIG. 2A, and one bioreactor over another heating element, such as in the case of bioreactor 274 located over heating element 284 in FIG. 2B. Moreover, additional sensors, beyond those depicted in FIGS. 2A-2C, can be used in a bioreactor. In one embodiment, an optical density sensor can be used to determine a culture concentration. For example, an optical density sensor can measure cell concentration of embryonic suspensor masses in the bioreactor. An optical density sensor may not operate ideally under all conditions, such as with large aggregates of embryonic suspensor masses in a bioreactor, but can be used in certain conditions, such as with smaller aggregates of embryonic suspensor masses.

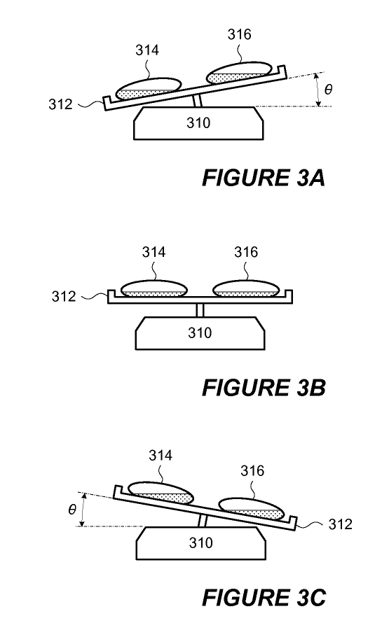

FIGS. 3A, 3B, and 3C depict side views of a rocker 310 with a pan 312 and two bioreactors 314 and 316 located on pan 312. In the view depicted in FIG. 3A, the right side of pan 312 is raised such that the pan 312 is at an angle θ with respect to horizontal. In the view depicted in FIG. 3B, the pan 312 is substantially horizontal. To arrive at the view depicted in FIG. 3B from the view depicted in FIG. 3A, the rocker 310 can rotate the pan 312 until the pan 312 is substantially horizontal. The rocker can continue to rotate the pan 312 until it arrives at the point depicted in FIG. 3C. In the view depicted in FIG. 3C, the left side of pan 312 is raised such that the pan 312 is at the angle θ with respect to horizontal. The rocker 310 can be configured to rock the pan 312 back and forth from the position depicted in FIG. 3A to the position depicted in FIG. 3C at a particular rate.

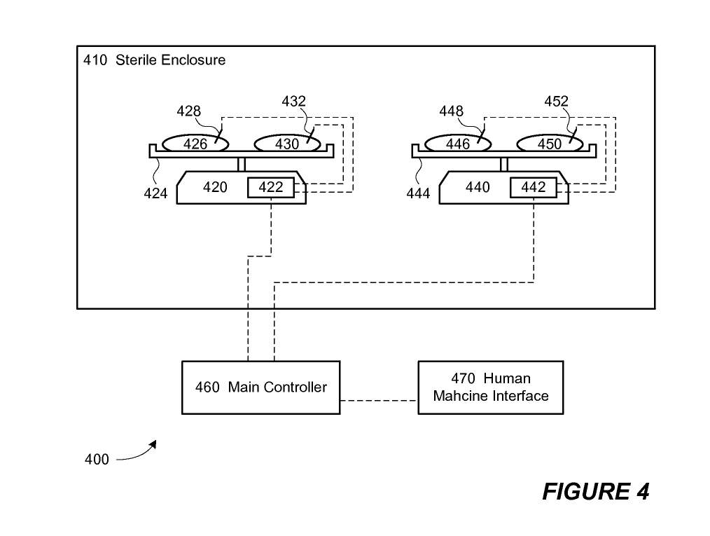

FIG. 4 depicts a bioreactor system 400 that includes control of multiple rockers. Bioreactor system 400 includes a sterile enclosure 410 that has two rockers 420 and 440. Rocker 420 includes a controller 422 and a pan 424. Bioreactor 426 is located on the left side of pan 424 and includes a temperature sensor 428. Bioreactor 430 is located on the right side of pan 424 and includes a temperature sensor 432. Signals from temperature sensors 428 and 432 can be sent to controller 422. Rocker 440 includes a controller 442 and a pan 444. Bioreactor 446 is located on the left side of pan 444 and includes a temperature sensor 448. Bioreactor 450 is located on the right side of pan 444 and includes a temperature sensor 452. Signals from temperature sensors 448 and 452 can be sent to controller 442.

Bioreactor system 400 can also include a main controller 460 and a human machine interface 470. Main controller 460 can be connected to each of controllers 422 and 442. Main controller 460 can send indications of a particular angle, a particular rocking speed, and a particular temperature to each of controllers 422 and 442. Those particular angles, rocking speeds, and temperatures can be entered by a user unto human machine interface 470 and communicated from human machine interface 470 to main controller 460. After controller 422 receives a particular angle, a particular rocking speed, and a particular temperature from main controller 460, the controller 422 can rock the pan 424 back and forth based on the particular angle and at the particular rocking speed, and the controller 422 can regulate the temperature of one or more heating elements in the pan 424 based on the particular temperature and the signals received from the temperature sensors 428 and 432. Similarly, after controller 442 receives a particular angle, a particular rocking speed, and a particular temperature from main controller 460, the controller 442 can rock the pan 444 back and forth based on the particular angle and at the particular rocking speed, and the controller 442 can regulate the temperature of one or more heating elements in the pan 444 based on the particular temperature and the signals received from the temperature sensors 448 and 452. It should be noted that the main controller 460 can send different angles, rocking rates, and temperatures to controller 422 and controller 442. Thus, the two rockers can operate independently under different conditions. The controllers 422 and 442 can also communicate information back to the main controller 460. For example, the controllers 422 and 442 can communicate, to main controller 460, indications of actual conditions of rockers 420 and 440, such as rocking angles, rocking speeds, and temperatures of rockers 420 and 440. The main controller 460 can send such indications of actual conditions to human machine interface 470 to be displayed so that a user can be informed of actual conditions.

While FIGS. 2A-2C and FIG. 4 depicts heating elements that are a part of rockers, any type of heating element can be configured to provide heat to a bioreactor. A heating element can be controlled by a heating controller. A heating controller can be part of a rocker (as described with respect to FIG. 4), a part of a main controller for the bioreactor system, an independent controller dedicated to control heating elements, and the like. The heating element can be configured to control the heating element based on signals received from temperature sensors. The heating element can be configured to control the heating element based on a particular temperature.

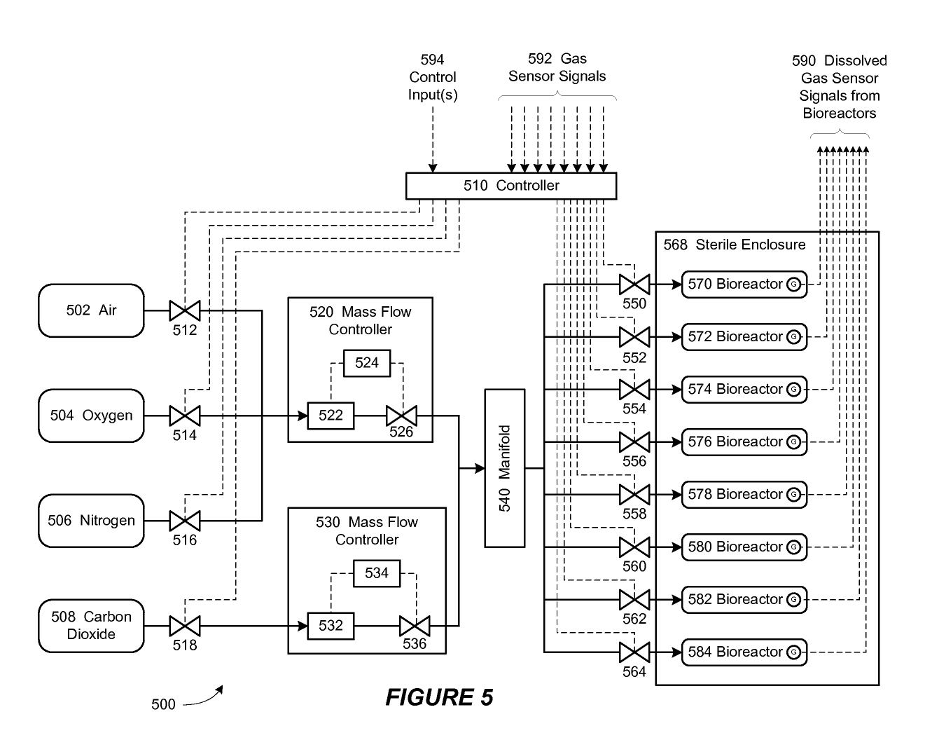

FIG. 5 depicts an embodiment of a gas source and control system 500. The gas source and control system 500 can include a source of air 502, a source of oxygen 504, a source of nitrogen 506, and a source of carbon dioxide 508. The gases leaving sources 502, 504, 506, and 508 can be controlled by gas source valves 512, 514, 516, and 518, respectively. The gas source valves 512, 514, 516, and 518 can be controlled by a controller 510. The gases that pass through gas source valves 512, 514, 516, and 518 can be fed into one of mass flow controller 520 and mass flow controller 530. In the embodiment shown in FIG. 5, gases from the source of air 502, the source of oxygen 504, and the source of nitrogen 506 are fed into mass flow meter 522 and to valve 526. Signals generated by mass flow meter 522 are sent to controller 524, and controller 524 controls valve 526 based on the signals generated by mass flow meter 522. Similarly, gases from the source of carbon dioxide 508 are fed into mass flow meter 532 and to valve 536. Signals generated by mass flow meter 532 are sent to controller 534, and controller 534 controls valve 536 based on the signals generated by mass flow meter 532. While the embodiment shown in FIG. 5 depicts three gas lines being fed into one mass flow controller 520 and one gas line being fed into another mass flow controller, other configurations, other configurations of gas lines and flow control meters are possible.

The gases that flow through valves 526 and 536 are sent to a single manifold 540. The single manifold can allow all of the gases exiting the mass flow controllers 520 and 530 to pass through a single pathway. The manifold 540 can include a humidifier that controls the amount of humidity in the gases. The manifold 540 is connected to each of gas valves 550, 552, 554, 556, 558, 560, 562, and 564, in parallel. Gas valves 550, 552, 554, 556, 558, 560, 562, and 564 are connected to bioreactors 570, 572, 574, 576, 578, 580, 582, and 584, respectively, that are located inside of sterile enclosure 568. Gas valves 550, 552, 554, 556, 558, 560, 562, and 564 are controlled independently by controller 510. The controller 510 can be configured to open one of gas valves 550, 552, 554, 556, 558, 560, 562, and 564 at a time in a cyclical manner. For example, controller 510 can open gas valve 550 for a period of time, such as 1 minute, close gas valve 550, open gas valve 552 for the period of time, close gas valve 552, and so forth, until each of gas valves 550, 552, 554, 556, 558, 560, 562, and 564 has been opened once. At that point, the controller 510 may return to opening gas valve 550 and start the cycle all over again. In this way, gases can be inserted into each of the eight bioreactors 570, 572, 574, 576, 578, 580, 582, and 584 in a cyclical pattern using the same gas sources 502, 504, 506, and 508, and the same mass flow controllers 520 and 530. The gases may mix somewhat in the path from the gas sources 502, 504, 506, and 508 to one of the bioreactors 570, 572, 574, 576, 578, 580, 582, and 584; however, most of the mixing of the gases may also occur inside of the bioreactors 570, 572, 574, 576, 578, 580, 582, and 584. Moreover, sterility filters can be placed on the gas lines between gas valves 550, 552, 554, 556, 558, 560, 562, and 564 and bioreactors 570, 572, 574, 576, 578, 580, 582, and 584 or at the location where the gas lines between gas valves 550, 552, 554, 556, 558, 560, 562, and 564 enters the bioreactors 570, 572, 574, 576, 578, 580, 582, and 584. Such sterility filters can ensure that any gasses entering bioreactors 570, 572, 574, 576, 578, 580, 582, and 584 are sterile.

Each of the bioreactors 570, 572, 574, 576, 578, 580, 582, and 584 can include one or more dissolved gas sensors that generate dissolved gas sensor signals 590. In one embodiment, dissolved gas sensor signals 590 can be sent to a main controller and the main controller can send dissolved gas sensor signals 592 to controller 510. In another embodiment, dissolved gas sensor signals 590 can be sent directly to controller 510 in the form of dissolved gas sensor signals 592. The controller 510 can also receive one or more control input signals 594. The control input signals 594 can be sent by a human machine interface, by another controller, and the like. The control input signals 594 can indicate controls for the system, such as indications whether one or more of bioreactors 570, 572, 574, 576, 578, 580, 582, and 584 are operating at a particular time, an indication of particular dissolved gas set points, an indication of a cycling rates of the gas valves 550, 552, 554, 556, 558, 560, 562, and 564, and the like. Controller 510 can control the opening and closing of gas source valves 512, 514, 516, and 518 and of gas valves 550, 552, 554, 556, 558, 560, 562, and 564 based on the control input signals 594.

The controller 510 can coordinate the cycle of opening and closing of the gas valves 550, 552, 554, 556, 558, 560, 562, and 564 with the opening and closing of source gas valves 512, 514, 516, and 518. The controller 510 can determine which of source gas valves 512, 514, 516, and 518 to open, in which order, and for what portion of the time that one of gas valves 550, 552, 554, 556, 558, 560, 562, and 564 is open. The determination can be based on dissolved gas sensor signals 592 received by the controller 510. For example, if one of the dissolved gas sensor signals 592 indicates that the level of dissolved oxygen in bioreactor 574 is low, then controller 510 can open oxygen source gas valve 514 for a portion of time followed by opening the air source gas valve 512 for a portion of time such that oxygen and air enter the bioreactor 574 when gas valve 554 is open. Controller 510 may asynchronously open source gas valves 512, 514, 516, and 518 with the opening of gas valves 550, 552, 554, 556, 558, 560, 562, and 564 such that the gases released from source gas valves 512, 514, 516, and 518 will reach the appropriate one of gas valves 550, 552, 554, 556, 558, 560, 562, and 564 when open. In another example, if one of the dissolved gas sensor signals 592 indicates that the level of dissolved oxygen in bioreactor 578 is high, then controller 510 can open nitrogen source gas valve 516 for a portion of time followed by opening the air source gas valve 512 for a portion of time such that nitrogen and air enter the bioreactor 578 when gas valve 558 is open.

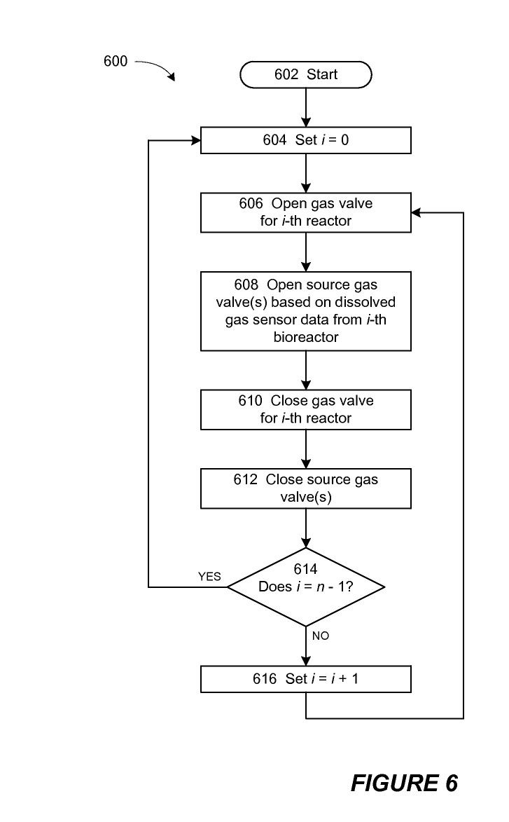

FIG. 6 depicts an embodiment of a method 600 of providing gases to bioreactors in a cyclical format. The method starts at block 602. At block 604, a variable i is set to 0. At block 606, the i-th gas valve corresponding to the i-th bioreactor can be opened. For example, in the case of the system 500 depicted in FIG. 5, bioreactor 570 can be the 0th bioreactor and, at block 606, the valve 550 corresponding to bioreactor 570 can be opened. At block 608, one or more source gas valves can be opened based on dissolved gas sensor data from the i-th bioreactor. For example, in the case of the system 500 depicted in FIG. 5, one or more of source gas valves 512, 514, 516, and 518, can be opened based on data from dissolved gas sensors 592. As discussed above, the one or more of source gas valves can be opened asynchronously with the i-th gas valve corresponding to the i-th bioreactor so that gases released through the one or more of source gas valves will pass through the i-th gas valve when the latter is open. The source gas valves can be opened in a particular order, such as opening one gas valve for a period of time and then opening a second gas valve after the period of time is over. At block 610, the i-th gas valve corresponding to the i-th bioreactor can be closed. At block 612, the one or more source gas valves can be closed.

At block 614 a determination can be made whether the variable i is equal to one less than a number n of bioreactors. For example, in the case of the system 500 depicted in FIG. 5, there are eight bioreactors 570, 572, 574, 576, 578, 580, 582, and 584. If the variable i has a value of seven, that means that a complete cycle through each of bioreactors 570, 572, 574, 576, 578, 580, 582, and 584 has been completed. In that case, the method returns back to block 604 where the variable i is reset to zero and the cycle restarts. However, if the variable i has a value less than seven, that means that the method has not yet cycled through each of the bioreactors 570, 572, 574, 576, 578, 580, 582, and 584. In such a case, the method proceeds to block 616 where the variable i is incremented up by one. At that point, the method returns back to block 606 where the i-th gas valve corresponding to the i-th bioreactor (i.e., the next bioreactor in the cycle) can be opened. Using the method depicted in FIG. 6, multiple gas sources can be connected independently to multiple bioreactors and the multiple bioreactors can be fed gases from the multiple gas sources in a cyclical manner.

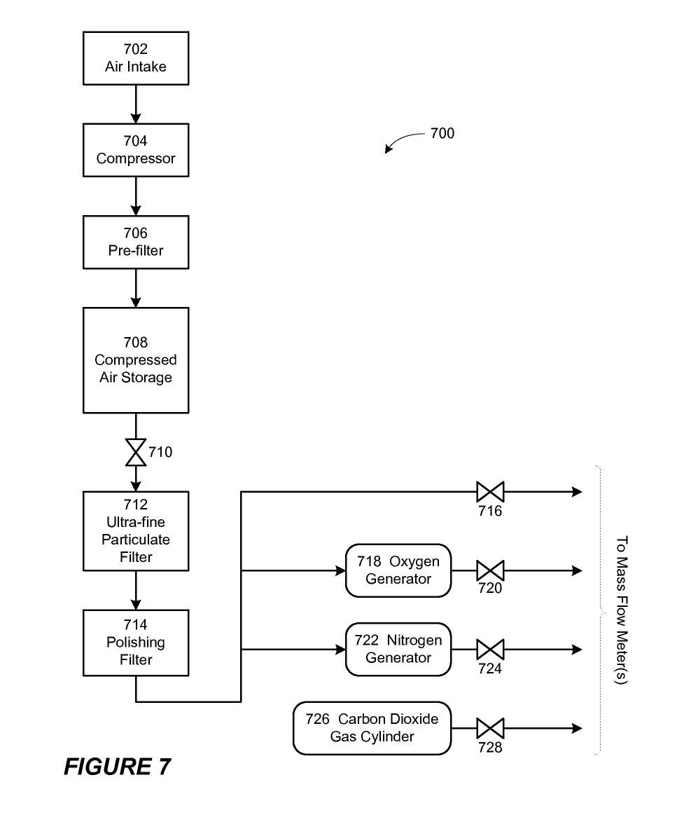

FIG. 7 depicts an embodiment of a system 700 of gas sources. The system 700 includes an air intake 702 and a compressor 704 configured to compress the air. The compressor 704 can include an engine or motor, such as an electrical motor, a gas engine, and the like, that can compress the air to a high pressure. The compressed air can be passed through a pre-filter 706 and stored in compressed air storage 708, such as a gas cylinder, a gas holder, and the like. The pre-filter 706 can remove particulate from the air compressed by compressor 704. A valve 710 can be located after the compressed air storage 708 to control the amount of compressed air that is released from the compressed air storage 708. After pass through valve 710, the air can pass through an ultra-fine particulate filter 712. The ultra-fine particulate filter 712 can remove small particulates from the air. After pass through ultra-fine particulate filter 712, the air can pass through a polishing filter 714. The polishing filter 714 can remove volatile organic compounds from the air. After pass through polishing filter 714, the air can be considered sufficiently clean for use by the remaining components of the system 700, such as oxygen generator 718 and nitrogen generator 722. While the air that has passed through polishing filter 714 may not be completely sterile, sterility filters can be used at the point where gasses flow into individual bioreactors.

The air exiting the polishing filter 714 can be passed to a valve 716 that controls air being fed to one or more flow meters. Valve 716 can function in a similar manner to the function of valve 512 described above with respect to FIG. 5. The air exiting the polishing filter 714 can also be passed to oxygen generator 718. Oxygen generator 718 can be configured to separate oxygen from the air and allow a gas containing a high percentage of oxygen to pass to valve 720 that controls oxygen being fed to one or more flow meters. Oxygen separation can be performed using a number of methods, such as cryogenic distillation, membrane pressure swing adsorption, and vacuum pressure swing adsorption, and the like. Valve 720 can function in a similar manner to the function of valve 514 described above with respect to FIG. 5. The air exiting the polishing filter 714 can also be passed to nitrogen generator 722. Nitrogen generator 722 can be configured to separate nitrogen from the air and allow a gas containing a high percentage of nitrogen to pass to valve 724 that controls nitrogen being fed to one or more flow meters. Nitrogen separation can be performed using a number of methods, such as cryogenic distillation, membrane pressure swing adsorption, and vacuum pressure swing adsorption, and the like. Valve 724 can function in a similar manner to the function of valve 516 described above with respect to FIG. 5. Gas sources can also include finite gas sources, such as the carbon dioxide gas cylinder 726. Carbon dioxide gas cylinder 726 can be connected to valve 728 that controls carbon dioxide being fed to one or more flow meters.

Each of the gas sources used in a bioreactor system can be either continuous gas sources, such as the sources of air, oxygen, and nitrogen depicted in FIG. 7, or finite gas sources, such as the source of carbon dioxide depicted in FIG. 7. The determination of whether to use a continuous gas source or a finite gas source for a particular type of gas may be based on a number of factors, such as cost to operate a continuous source, cost to purchase finite sources, frequency of replacing a finite gas source, desire for uninterrupted operation of a bioreactor system, and the like. For example, if relatively low amounts of carbon dioxide are used by a bioreactor system, it may be advantageous to use a finite gas source of carbon dioxide. In another example, if relatively high amounts of oxygen are used in a bioreactor system that operates continuously for weeks or longer, it may be advantageous to use a continuous gas source of oxygen.

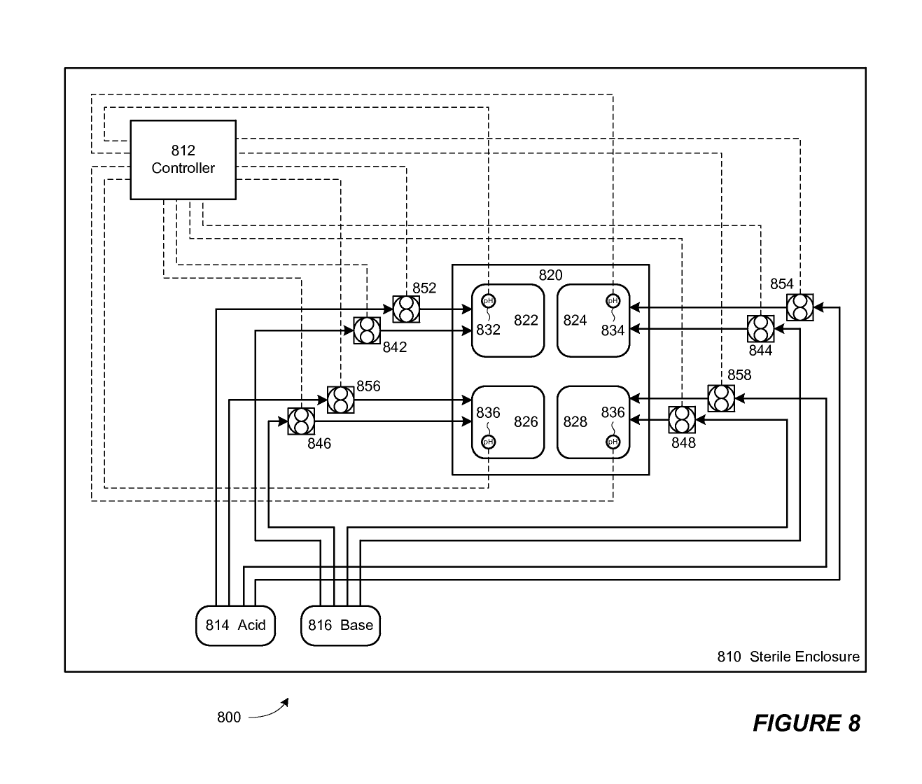

FIG. 8 depicts an example of a pH control system inside of a bioreactor system 800. The bioreactor system 800 includes a sterile enclosure 810, a controller 812, a source of acidic material 814, and a source of basic material 816. While the controller 812, the source of acidic material 814, and the source of basic material 816 are depicted as being within the sterile enclosure 810, it is possible for the controller 812, the source of acidic material 814, and the source of basic material 816 to be located outside of the sterile enclosure 810. It may be advantageous for the source of acidic material 814 and the source of basic material 816 to be kept within the sterile enclosure 810 to avoid any contaminating substance from entering the acidic material in the source of acidic material 814 or the basic material in the source of basic material 816. The controller 812 can be located outside of the sterile enclosure 810 without as much concern about contamination. However, if controller 812 is located outside of the sterile enclosure 810, wiring connecting the controller 812 to various components inside of the sterile enclosure 810 would need to pass through a wall of the sterile enclosure 810 possibly leaving a hole for outside air to enter the sterile enclosure 810.

The bioreactor system 800 also include a rocker pan 820, which is shown from a top view. In the particular bioreactor system 800 depicted in FIG. 8, four bioreactors 822, 824, 826, and 828 are on the rocker pan 820. The bioreactors 822, 824, 826, and 828 can include pH sensors 832, 834, 836, and 838 that sense a level of pH within bioreactors 822, 824, 826, and 828, respectively. Signals are sent from each of the bioreactors 822, 824, 826, and 828 to the controller 812. Controller 812 can also be in communication with peristaltic pumps 852, 854, 856, 858, 862, 864, 866, and 868. Peristaltic pumps 852, 854, 856, and 858 can be configured to pump basic material from the source of basic material 816 into bioreactors 822, 824, 826, and 828, respectively. Peristaltic pumps 842, 844, 846, and 848 can be configured to pump acidic material from the source of acidic material 814 into bioreactors 822, 824, 826, and 828, respectively.

The controller 812 can determine, based on the signals received from the pH sensors 832, 834, 836, and 838, whether pH in each bioreactor meets a particular pH set point. In the case where a particular pH set point is a predetermined range of pH values, if one of the bioreactors 822, 824, 826, and 828 is not within the predetermined range of pH values, the controller 812 can instruct one of the peristaltic pumps 852, 854, 856, 858, 862, 864, 866, and 868 to pump an amount of basic material or acidic material into the one of the bioreactors 822, 824, 826, and 828 that is not within a predetermined range. For example, a desired range of pH values for totipotent plant tissue multiplication can be between 5.0 and 5.7. If the controller 812 receives a signal from pH sensor 834 indicating that the level of pH in bioreactor 824 is below 5.0, the controller 812 can send a signal to peristaltic pump 844 to pump an amount of basic material from the source of basic material into bioreactor 824. The controller 812 may determine the amount of basic material to be pumped into bioreactor 824 based on how far below the predetermined range the level of pH is in bioreactor 824, based on a rate of change of pH within bioreactor 824, or any other calculation. Using the same desired range of pH values, the controller 812 may receive a signal from pH sensor 836 indicating that the level of pH in bioreactor 826 is above 5.7. The controller 812 can send a signal to peristaltic pump 856 to pump an amount of acidic material from the source of acidic material into bioreactor 826. The controller 812 may determine the amount of acidic material to be pumped into bioreactor 826 based on how far above the predetermined range the level of pH is in bioreactor 826, based on a rate of change of pH within bioreactor 826, or any other calculation. Similarly, a pH set point can be a particular pH value. In this case, the controller 812 can instruct one of the peristaltic pumps 852, 854, 856, 858, 862, 864, 866, and 868 to pump an amount of basic material or acidic material into the one of the bioreactors 822, 824, 826, and 828 to drive the level of pH within one of the bioreactors 822, 824, 826, and 828 to a particular pH value.

The bioreactor system 800 depicted in FIG. 8 could be modified to have capacity for up to eight bioreactors operating at one time. For example, the sterile enclosure 810 could include two rockers that each holds up to four bioreactors. In this example, the source of acidic material 814 could be connected to a first set of eight peristaltic pumps, where each one of the first set of peristaltic pumps could be configured to pump acidic material into one of the eight bioreactors. The source of basic material 816 could be connected to a second set of eight peristaltic pumps, where each one of the second set of peristaltic pumps could be configured to pump basic material into one of the eight bioreactors. A single controller 812 could be used to control each of the first set of peristaltic pumps and each of the second set of peristaltic pumps, or multiple controllers could be used to control the first and second sets of peristaltic pumps. For example, the bioreactor system 800 could include one controller for each of the bioreactors 822, 824, 826, and 828, and each of the controllers could control the peristaltic pumps that pump acidic material and basic material for the particular bioreactor corresponding to the controller.

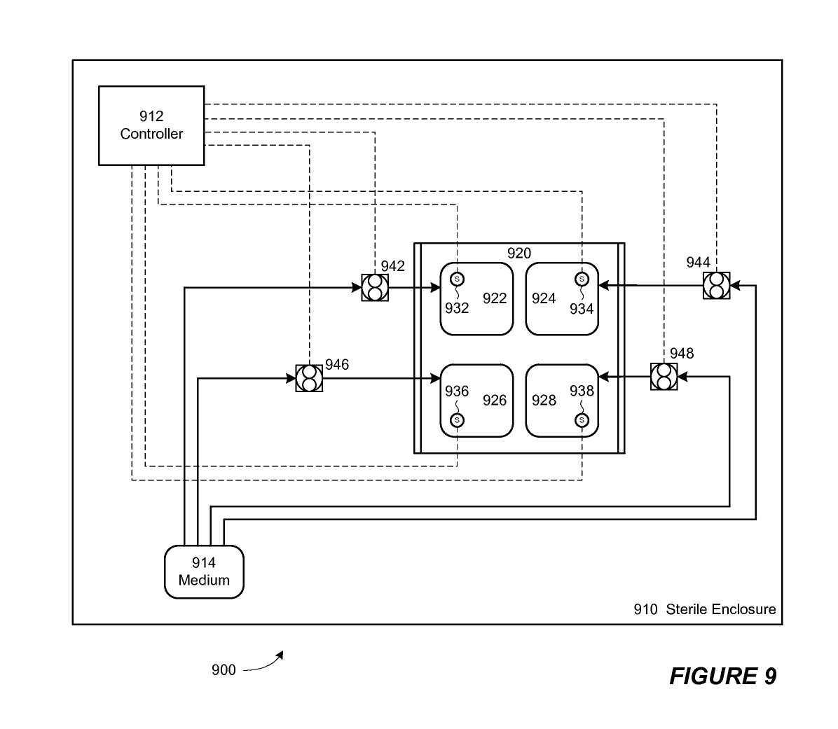

FIG. 9 depicts a bioreactor system 900 with a control system for controlling an amount of multiplication medium that is fed into bioreactors. The rate of totipotent plant tissue multiplication in a bioreactor may be affected by the amount of liquid multiplication medium is in the bioreactor. As totipotent plant tissue multiplication proceeds, it uses up portions of the multiplication medium in the bioreactor, including the sugars in the multiplication medium. Thus, whether multiplication medium should be added to a bioreactor can be determined based on a percentage of sugar in the bioreactor.

The bioreactor system 900 includes a sterile enclosure 910, a controller 912, and a source of multiplication medium 914. While the controller 912 and the source of multiplication medium 914 are depicted as being within the sterile enclosure 910, it is possible for the controller 912 and the source of multiplication medium 914 to be located outside of the sterile enclosure 910. It may be advantageous for the source of a multiplication medium 914 to be kept within the sterile enclosure 910 to avoid any contaminating substance from entering the medium in the source of multiplication medium 914. The controller 912 can be located outside of the sterile enclosure 910 without as much concern about contamination. However, if controller 912 is located outside of the sterile enclosure 910, wiring connecting the controller 912 to various components inside of the sterile enclosure 910 would need to pass through a wall of the sterile enclosure 910 possibly leaving a hole for outside air to enter the sterile enclosure 910.

The bioreactor system 900 also include a rocker pan 920, which is shown from a top view. In the particular bioreactor system 900 depicted in FIG. 9, four bioreactors 922, 924, 926, and 928 are on the rocker pan 920. The bioreactors 922, 924, 926, and 928 can include sugar sensors 932, 934, 936, and 938 (e.g., Brix sensors) that sense a level of sugar concentration within bioreactors 922, 924, 926, and 928, respectively. Signals are sent from each of the bioreactors 922, 924, 926, and 928 to the controller 912. Controller 912 can also be in communication with peristaltic pumps 952, 954, 956, and 958. Peristaltic pumps 952, 954, 956, and 958 can be configured to multiplication medium from the source of multiplication medium 914 into bioreactors 922, 924, 926, and 928, respectively.

The controller 912 can determine, based on the signals received from the sugar sensors 932, 934, 936, and 938, whether sugar concentration in one of the bioreactors 922, 924, 926, and 928 meets a sugar set point. In an example where a sugar set point is a low level of sugar concentration, if a sugar concentration in one of the bioreactors 922, 924, 926, and 928 is below the low level of sugar concentration, the controller 912 can instruct one of the peristaltic pumps 952, 954, 956, and 958 to pump an amount of multiplication medium into the one of the bioreactors 922, 924, 926, and 928 that is below the predetermined level. For example, it may be desirable to keep a sugar concentration in a bioreactor above 3% by weight. If the controller 912 receives a signal from sugar sensor 934 indicating that the sugar concentration in bioreactor 924 is below 3%, the controller 912 can send a signal to peristaltic pump 944 to pump an amount of multiplication medium from the source of multiplication medium 914 into bioreactor 924. The controller 912 may determine the amount of multiplication medium to be pumped into bioreactor 924 based on how far below predetermined level the sugar concentration is in bioreactor 924, based on a rate of change of sugar concentration within bioreactor 924, or any other calculation. Similarly, the sugar set point can be a range of sugar concentration. In this case, the controller 912 can instruct one of the peristaltic pumps 952, 954, 956, and 958 to pump an amount of multiplication medium into the one of the bioreactors 922, 924, 926, and 928 to maintain the sugar concentration level within one of the bioreactors 822, 824, 826, and 828 within that range of sugar concentration.

Alternatively, a medium dosing system can be an off-line system. In an off-line medium dosing system, the bioreactors 922, 924, 926, and 928 may not include sugar sensors 932, 934, 936, and 938. However, the controller 912 can be configured to control peristaltic pumps 952, 954, 956, and 958 to pump a particular dose of medium into each of the bioreactors 922, 924, 926, and 928 at a particular dosing rate. The culture in each of the bioreactors 922, 924, 926, and 928 can be sampled on occasion and the sugar concentration of the samples can be determined. A user can adjust the dose amount and/or the dosing rate controlled by the controller 912 based on the sugar concentration of the samples. The adjustments of the dose amount and/or the dosing rate can be received as inputs into a human machine interface (not shown) and sent to the controller 912. Alternatively, a user could determine a sugar concentration of the sampled culture and enter that determined sugar concentration into the human machine interface. Controller 912 can be configured to calculate a particular dose or doses of medium into each of the bioreactors 922, 924, 926, and 928 at a particular dosing rate based on the sugar concentrate input by the user. The controller 912 can control peristaltic pumps 952, 954, 956, and 958 to pump a particular dose or doses of medium into each of the bioreactors 922, 924, 926, and 928 at a particular dosing rate based on the calculations by the controller 912. Moreover, medium dosing can be added at non-linear amounts and rates, such as by exponentially increasing dose amounts, exponentially increasing dose rates, and the like.

The bioreactor system 900 depicted in FIG. 9 could be modified to have capacity for up to eight bioreactors operating at one time. For example, the sterile enclosure 910 could include two rockers that each hold up to four bioreactors. In this example, the source of multiplication material 914 could be connected to a set of eight peristaltic pumps, where each one of the set of peristaltic pumps could be configured to pump multiplication material into one of the eight bioreactors. A single controller 912 could be used to control each of the peristaltic pumps or multiple controllers could be used to control each of the peristaltic pumps. For example, the bioreactor system 900 could include one controller for each of the bioreactors 922, 924, 926, and 928, and each of the controllers could control the peristaltic pump that pumps medium for the particular bioreactor corresponding to the controller.

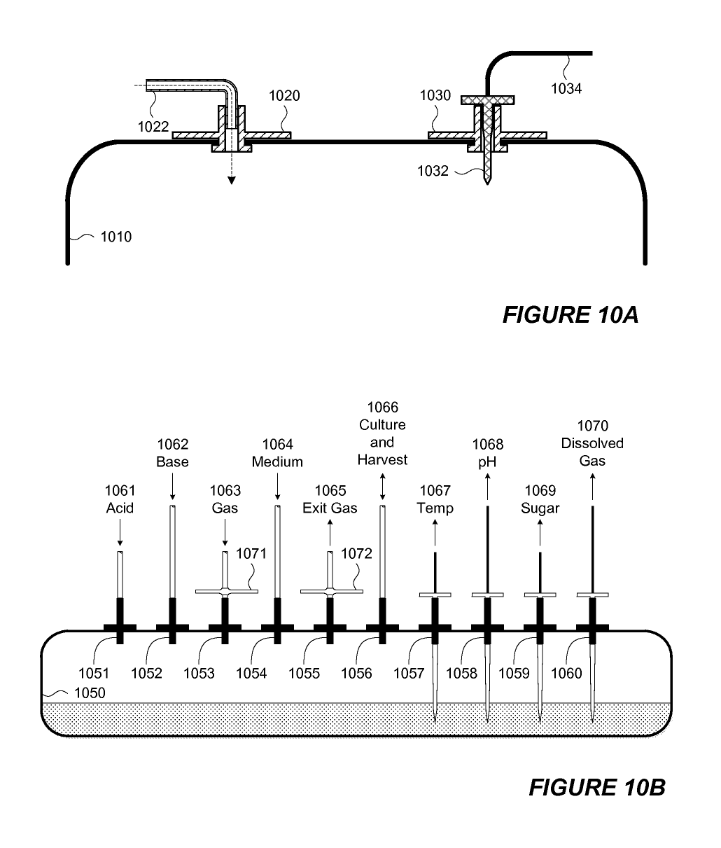

FIGS. 10A and 10B depict examples of ports used with bioreactors. FIG. 10A depicts a cross-sectional view of a portion of a bioreactor 1010. Bioreactor 1010 can be in the form of a bag and can provide a sterile enclosure. To maintain the sterile environment within the bioreactor 1010, the bioreactor 1010 can include ports 1020 and 1030. In FIG. 10A, cross-sectional views of ports 1020 and 1030 are depicted. Ports 1020 and 1030 can be attached to the side of bioreactor 1010 in a way that prevents contamination of the bioreactor 1010 from the environment outside of the bioreactor 1010. As shown, port 1020 can be configured to receive a portion of tubing 1022. Once port 1020 has received tubing 1022, liquid or gases can be passed through the tubing and into the bioreactor 1010. The port 1020 can be configured to seal itself when tubing 1022 is withdrawn such that pulling tubing 1022 out of port 1020 does not expose the interior of the bioreactor 1010 to the outer environment. As shown, port 1030 can be configured to receive a sensor 1032. Sensor 1032 can sense a condition within the bioreactor 1010 and send a signal along connection 1034. Connection 1034 can be an electrical connection, such as a wire, an optical connection, such as a fiber optic, or any other connection that can carry a signal. Alternatively, sensor 1032 could be configured to pass a signal wirelessly. The port 1030 can be configured to seal itself when sensor 1032 is withdrawn such that pulling the sensor 1032 out of port 1030 does not expose the interior of the bioreactor 1010 to the outer environment.

FIG. 10B depicts a cross-sectional view of a portion of a bioreactor 1050. Bioreactor 1050 includes ports 1051-1060. Port 1051 can be configured to receive tubing 1061 that can pass an acidic material into the bioreactor 1050. As described above, tubing 1061 can be connected to a peristaltic pump that can control an amount of acidic material that is inserted into the bioreactor 1050. Port 1052 can be configured to receive tubing 1062 that can pass a basic material into the bioreactor 1050. As described above, tubing 1062 can be connected to a peristaltic pump that can control an amount of basic material that is inserted into the bioreactor 1050. Port 1053 can be configured to receive tubing 1063 that can pass gasses into the bioreactor 1050. As described above, tubing 1063 can be connected to a valve that is cyclically opened to allow gases to be inserted into the bioreactor 1050. A sterilization filter 1071 can be placed along tubing 1063, at the point that tubing 1063 meets port 1053, or along port 1053. The sterilization filter 1071 can ensure that any gasses passing from tube 1063 into bioreactor 1050 are sterile. Port 1054 can be configured to receive tubing 1064 that can pass a multiplication medium into the bioreactor 1050. As described above, tubing 1064 can be connected to a peristaltic pump that can control an amount of multiplication medium that is inserted into the bioreactor 1050.

Port 1055 can be configured to receive tubing 1065 that can allow gas to exit the bioreactor 1050. A sterilization filter 1072 can be placed along tubing 1065, at the point that tubing 1065 meets port 1055, or along port 1055. The sterilization filter 1072 can ensure that any gasses passing back from tube 1065 into bioreactor 1050 are sterile. Port 1056 can be configured to receive tubing 1066 that can be used to pass culture into and harvest culture from the bioreactor 1050. Culture can be passed into the bioreactor 1050 when the bioreactor is starting up (i.e. inoculated) or at any point during totipotent plant tissue multiplication. All of the culture from the bioreactor 1050 can be harvested at once. Alternatively, some of the culture can be harvested at any given time during totipotent plant tissue multiplication. For example, if the amount of material in the bioreactor 1050 is approaching the maximum volume of the bioreactor 1050, a portion of the culture can be harvested out of the bioreactor 1050 to allow the specimen to continue growth continues in the bioreactor 1050.

Port 1057 can be configured to receive sensor 1067 that can sense temperature in the bioreactor 1050. Sensor 1067 can send a signal indicative of the temperature to one or more controllers. Port 1058 can be configured to receive sensor 1068 that can sense a level of pH in the bioreactor 1050. Sensor 1068 can send a signal indicative of the level of pH to one or more controllers. Port 1059 can be configured to receive sensor 1069 that can sense a level of sugar concentration in the bioreactor 1050. Sensor 1069 can send a signal indicative of the level of sugar concentration to one or more controllers. Port 1060 can be configured to receive sensor 1070 that can sense a level of dissolved oxygen or other gas in the bioreactor 1050. Sensor 1070 can send a signal indicative of the level of dissolved oxygen or other gas to one or more controllers.

Each bioreactor in a bioreactor system can include a number of ports, such as the bioreactor 1050 shown in FIG. 10B. If a bioreactor has sufficient ports to connect the bioreactor to the appropriate system for automatic monitoring and control of the conditions in the bioreactor, then totipotent plant tissue multiplication can continue inside of the bioreactor with minimal user intervention.

In addition, when a bioreactor is located in a sterile enclosure, having a number of ports on the bioreactor can allow for independent adjustment of the sensors and/or tubing attached to the ports. For example, a bioreactor can have a one or more sensors inserted into ports in the bioreactor. The one or more sensors can include a dissolved gas sensor, a sugar concentration sensor, a pH sensor, an optical density sensor, a temperature sensor, and the like. The bioreactor can also have tubing attached to one or more sensors. The tubing can be part of a matter insertion system, such as a medium dosing system that is configured to insert media into the bioreactor, a pH control system that is configured to insert acidic material and/or basic material into the bioreactor, a gas control system that is configured to transfer gasses into the bioreactor, a culture transfer system configured to transfer culture into the bioreactor, and the like. The bioreactor can also have one or more ports that are available for infrequent use, such as a port to which tubing from a culture harvest system can be attached in order to harvest culture from the bioreactor.

A bioreactor can have a number of sensors and/or tubing attached to ports during totipotent plant tissue multiplication. If one of the port attachments needs adjustments—such as removal of a sensor from a port, disconnection of tubing from a port, connection of tubing to a port, and the like—the port attachment adjustment can be made inside of the sterile enclosure to preserve the sterile environment within the bioreactor and without affecting operation of the other port attachments. For example, a sensor can be inserted in a first port and tubing from a matter insertion system can be connected to a second port. If the sensor needs to be removed for some reason, such as the sensor is defective or no longer needed in the bioreactor, the sensor can be removed from the first port without affecting operation of the matter insertion system. Similarly, if the tubing needs to be removed from the second port for some reason, the tubing can be disconnected from the second port without affecting operation of the second port. In both cases, the removal of the sensor and the disconnection of the tubing can be accomplished while the totipotent plant tissue multiplication process continues inside of the bioreactor. The location of the bioreactor inside of the sterile enclosure can minimize or eliminate the risk of contaminating the environment inside of the bioreactor.

In another example, a bioreactor can have at least one sensor inserted into a first port and a first tubing connected to a second port. The first tubing can be a part of a matter insertion system. Totipotent plant tissue multiplication can occur in the bioreactor with the at least one sensor inserted in the first port and the first tubing connected to the second port. The bioreactor can include a third port. When culture is to be removed from the bioreactor, a second tubing from a culture harvest system can be connected to the third port. The second tubing can be connected to the third port without removing the bioreactor from the sterile enclosure and without disturbing operation of the at least one sensor and the at least one sensor inserted in the first port and the first tubing connected to the second port. In this manner, culture can be harvested and/or sampled from the bioreactor without having to remove the at least one sensor from the first port and without having to disconnect the first tubing from the second port.

Obviating a need to remove sensors from ports of the bioreactor and disconnecting tubing from matter insertion systems can have a number of advantages. In normal operation, a bioreactor can have a number of sensors and a number of tubings connected during totipotent plant tissue multiplication. The time it takes a technician to remove and/or disconnect all of the sensors and detach all of the tubings can be significant. If the harvesting and/or sampling of culture from the bioreactor can be accomplished without removing and/or disconnecting all of the sensors and detaching all of the tubings, significant amounts of time can be saved when harvesting and/or sampling culture from the bioreactor. This saves labor time that it would take a technician to harvest and/or sample culture from the bioreactor, and it also increases the amount of time that the bioreactor can be available for totipotent plant tissue multiplication. As the number of bioreactors in a sterile enclosure increases, so too does the time savings that can be achieved by not needing to remove any of the bioreactors from the sterile enclosure when making changes to port attachments.

A sterile enclosure can also aid in maintaining sterility of sources of matter for matter insertion systems. Sources of matter can include a source of medium for a medium dosing system, a source of acidic material and/or a source of basic material for a pH control system, and the like. Before tubing is connected between a source of matter and a port of a bioreactor, the source of matter can be placed inside of the sterile enclosure. While inside of the sterile enclosure, the source of matter can be opened (e.g., a port of the source of matter can be opened), a port on a bioreactor can be opened, and tubing can be connected between the source of matter and the port on the bioreactor. A pump, such as a peristaltic pump, can be placed along the tubing to pump matter from the source of matter into the bioreactor. The sterile enclosure may be large enough to store the source of matter indefinitely so that the source of matter remains inside of the sterile enclosure during totipotent plant tissue multiplication inside of the bioreactor. It may be advantageous to leave sources of matter inside of a sterile enclosure in cases where multiple bioreactors are used inside of a single sterile enclosure, as runs of totipotent plant tissue multiplication may be started or stopped at differed times in the bioreactors and bioreactors may be added to or removed from the sterile enclosure at different times. Alternatively, once the tubing connection is complete between the source of matter and the bioreactor, the source of matter can be removed from the sterile enclosure to the extent that the length of the tubing allows. In this latter example, moving the source of matter outside of the sterile enclosure would not raise significant risk of contamination of the source of matter or the bioreactor as the connection was established in a sterile environment.

Referring back to FIG. 1, an embodiment of bioreactor system 100 can be described. Bioreactor system 100 can include a sterile environment 102 that includes two rockers 106 and 110. Four bioreactors 104 can be placed on rocker 106 and four bioreactors 108 can be placed on rocker 110. The rockers 106 and 110 can be configured to continuously rock the bioreactors 104 and the bioreactors 108 to particular angles and particular speeds. Some or all of bioreactors 104 and 108 can include a temperature sensor that sends signals indicative of temperature in the bioreactors 104 and 108 to the rockers 106 and 110. The rockers can control a heating element in a pan of the rocker to regulate temperature in the bioreactors 104 and 108 to a particular temperature. The particular angles, particular speeds, and particular speeds can be sent to the rockers 106 and 110 from main controller 120 after being input into human machine interface 122 by a user.

The pH control system 112 can include a pH sensor in each of the bioreactors 104 and 108, a source of acidic material in the sterile enclosure 102, a source of basic material in the sterile enclosure 102, eight peristaltic pumps configured to pump acidic material from the source of acidic material to each of the bioreactors 104 and 108, eight peristaltic pumps configured to pump basic material from the source of basic material to each of the bioreactors 104 and 108, and a controller. The controller of the pH control system 112 can be the main controller 120 or a separate controller. The pH control system 112 can be configured to maintain pH in each of the bioreactors 104 and 108 based on a pH set point, such as maintaining the pH level within a predetermined range of pH or driving the level of pH in each of the bioreactors 104 and 108 to a particular level. The pH set point can be received by the human machine interface 122 from a user and fed to the main controller 120.

The medium dosing system 114 can include a sugar concentration sensor in each of the bioreactors 104 and 108, a source of medium in the sterile enclosure 102, eight peristaltic pumps configured to pump medium from the source of medium to each of the bioreactors 104 and 108, and a controller. The controller of the medium dosing system 114 can be the main controller 120 or a separate controller. The medium dosing system 114 can be configured to maintain a level of sugar concentration in each of the bioreactors 104 and 108 based on a sugar set point, such as maintaining the sugar level above, below, or at a particular level of sugar concentration. The particular level of sugar concentration can be received by the human machine interface 122 from a user and fed to the main controller 120.

The culture transfer and harvesting system 116 can include a source of culture, equipment configured to add culture to the bioreactors 104 and 108, a repository for harvested culture, and equipment configured to harvest culture from the bioreactors 104 and 108. Main controller 120 or another controller can automatically control the amount of culture transferred to and harvested from the bioreactors 104 and 108. The culture transfer and harvesting system 116 can also transfer to and harvested from the bioreactors 104 and 108 on a periodic or scheduled basis. The human machine interface 122 can be used to initiate or enter settings for control of the culture transfer and harvesting system 116.

The gas source and control system 118 can include sources of gas, one or more mass flow controller, a single manifold to receive all of the gas passing out of the one or more mass flow controllers, a gas source valve connected to each of the sources of gas, eight valves connected in parallel to the manifold and connected to each of the bioreactors 104 and 108, one or more dissolved gas sensors in each of the bioreactors 104 and 108, and one or more controllers. The sources of gas can be continuous or finite. The one or more mass flow controllers can control an amount of gas entering the manifold. The valves connecting the manifold to each of the bioreactors 104 and 108 can be configured to open in a cyclical fashion and the gas source valves can be controlled to permit a particular amount of different types of gas to enter each of the bioreactors 104 and 108. The gas source valves can be controlled based on signals from the one or more dissolved gas sensors in each of the bioreactors 104 and 108. The one or more dissolved gas sensors can sense any or all of the following in each of the bioreactors 104 and 108: a level of dissolved oxygen in the bioreactor, a level of carbon dioxide in the bioreactor, and a level of nitrogen in the bioreactor. The controller of the gas source and control system 118 can be the main controller 120, a separate controller, or some combination of the main controller 120 and one or more separate controllers.

The main controller 120 and the human machine interface 122 can take the form of a programmed computer that is configured to perform the functions of the main controller 120 and the human machine interface 122. The database 124 can store data about each trial of totipotent plant tissue multiplication in each bioreactor 104 and 108. The information stored in the database 124 can include operating conditions of the trial, the length of the trial, the yield of the trial, the quality of the totipotent plant tissue developed during the trial, and so forth. The controller 120 can automatically store such information in the database 124 or a user can initiate storage of information in the database 124 using human machine interface 122. The human machine interface 122 can also display information retrieved from the database 124. For example, the human machine interface 122 can display a chart showing historical yields for trials run under particular conditions, amounts of totipotent plant tissue developed during particular trials, and the like. The information in database 124 can also serve as the basis for determining preferred operating conditions for particular forms of totipotent plant tissue multiplication.

Each of the processes, methods, and algorithms described in the preceding sections may be embodied in, and fully or partially automated by, code modules executed by one or more computers or computer processors. The code modules may be stored on any type of non-transitory computer-readable medium or computer storage device, such as hard drives, solid state memory, optical disc, and/or the like. The processes and algorithms may be implemented partially or wholly in application-specific circuitry. The results of the disclosed processes and process steps may be stored, persistently or otherwise, in any type of non-transitory computer storage such as, e.g., volatile or non-volatile storage.

The various features and processes described above may be used independently of one another, or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

It will also be appreciated that various items are illustrated as being stored in memory or on storage while being used, and that these items or portions of thereof may be transferred between memory and other storage devices for purposes of memory management and data integrity. Alternatively, in other embodiments some or all of the software modules and/or systems may execute in memory on another device and communicate with the illustrated computing systems via inter-computer communication. Furthermore, in some embodiments, some or all of the systems and/or modules may be implemented or provided in other ways, such as at least partially in firmware and/or hardware, including, but not limited to, one or more application-specific integrated circuits (ASICs), standard integrated circuits, controllers (e.g., by executing appropriate instructions, and including microcontrollers and/or embedded controllers), field-programmable gate arrays (FPGAs), complex programmable logic devices (CPLDs), etc. Some or all of the modules, systems and data structures may also be stored (e.g., as software instructions or structured data) on a computer-readable medium, such as a hard disk, a memory, a network, or a portable media article to be read by an appropriate drive or via an appropriate connection. The systems, modules and data structures may also be transmitted as generated data signals (e.g., as part of a carrier wave or other analog or digital propagated signal) on a variety of computer-readable transmission media, including wireless-based and wired/cable-based media, and may take a variety of forms (e.g., as part of a single or multiplexed analog signal, or as multiple discrete digital packets or frames). Such computer program products may also take other forms in other embodiments. Accordingly, other computer system configurations are possible.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While certain example embodiments have been described, these embodiments have been presented by way of example only, and are not intended to limit the scope of the inventions disclosed herein. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

What is claimed:

1. A system for performing totipotent plant tissue multiplication, the system comprising:
a sterile enclosure;
a bioreactor in the sterile enclosure, the bioreactor comprising a plurality of ports;
at least one sensor inserted into a first port of the plurality of ports;
a matter insertion system configured to input matter into the bioreactor via a first tubing configured to be connected to a second port of the plurality of ports; and
wherein the at least one sensor can be removed from the first port during operation of the matter insertion system, and wherein first tubing can be disconnected from the second port during operating of the at least one sensor.

2. The system of claim 1, wherein the at least one sensor comprises one or more of a dissolved gas sensor, a sugar sensor, a pH sensor, an optical density sensor, or a temperature sensor.

3. The system of claim 1, wherein the at least one sensor is in communication with a controller and wherein the controller is in communication with the matter insertion system.

4. The system of claim 1, wherein the matter insertion system is configured to control input of the matter into the bioreactor based on signals from the at least one sensor.

5. The system of claim 1, wherein the matter comprises one or more of medium, acidic material, basic material, or gasses.

6. The system of claim 1, wherein the matter comprises gasses, and wherein the first tubing comprises a sterilization filter configured to sterilize gasses entering the bioreactor via the first tubing.

7. The system of claim 1, wherein the system further comprises:
a culture transfer system configured to either transfer culture into or harvest culture out of the bioreactor via a second tubing.

8. The system of claim 7, wherein the second tubing can be connected to a third port of the plurality of ports during operation of the at least one sensor and the matter insertion system.

9. The system of claim 8, wherein the culture transfer system comprises a pump.

10. The system of claim 9, wherein the pump is configured to pump culture from a culture source into the bioreactor via the second tubing.

11. The system of claim 9, wherein the pump is configured to pump culture out of the bioreactor via the second tubing and into a harvest container.

12. The system of claim 9, wherein the pump is configured to pump a sample of culture out of the bioreactor via the second tubing.

13. The system of claim 1, wherein the matter insertion system comprises a source of matter connected to the first tubing.

14. The system of claim 13, wherein the source of matter is located outside of the sterile enclosure when the first tubing is connected to the source of matter and the second port.

15. A method of performing totipotent plant tissue multiplication, the method comprising:
inserting at least one sensor into a first port of a bioreactor;
connecting a first tubing to a second port of the bioreactor, the first tubing being a part of a matter insertion system configured to input matter into the bioreactor via the first tubing;
performing totipotent plant tissue multiplication within the bioreactor while the bioreactor is located inside of a sterile enclosure; and
performing one or more of the following during totipotent plant tissue multiplication within the bioreactor:
removing the at least one sensor from the first port during operation of the matter insertion system, and
disconnecting the first tubing from the second port during operation of the at least one sensor.

16. The method of claim 15, further comprising:
attaching a second tubing to a third port of the bioreactor without affecting operation of the at least one sensor and the matter insertion system, the second tubing being a part of a culture transfer system configured to either transfer culture into or harvest culture out of the bioreactor via the second tubing.

17. The method of claim 16, further comprising:
transferring culture from the bioreactor to a harvest container via the second tubing while the harvest container and the bioreactor are located within the sterile enclosure.

18. The method of claim 17, further comprising:
closing the harvest container while is located within the sterile enclosure.

19. The method of claim 18, further comprising:
removing the closed harvest container from the sterile enclosure.

20. The method of claim 17, further comprising:
weighing the culture transferred from the bioreactor into the harvest container.

21. The method of claim 20, further comprising:
transferring a portion of the culture from the harvest container into the bioreactor via the second tubing after weighing the culture; and
performing totipotent plant tissue multiplication within the bioreactor using the portion of the portion of the culture transferred from the harvest container into the bioreactor.

22. The method of claim 15, further comprising:
controlling the matter insertion system during totipotent plant tissue multiplication within the bioreactor based on signals from the at least one sensor.

23. The method of claim 15, further comprising:
connecting the first tubing to a source of matter while the source of matter and the bioreactor are located in the sterile enclosure.

24. The method of claim 23, further comprising:
removing the source of matter from the sterile enclosure after the first tubing is connected to the source of matter and the second port of the bioreactor.
